(19) United States
(12) Patent Application Publication
(10) Pub. No.: US 2026/0224391 A1
(43) Pub. Date: Aug. 6, 2026

(54) Sleep Breathing Disorder Alleviation Device and Sleep Breathing Disorder Alleviation System

(71) Applicant: MD Staage Inc., Seoul (KR)

(72) Inventor: Seong Taek KIM, Seoul (KR)

(21) Appl. No.: 19/122,740

(22) PCT Filed: Oct. 19, 2023
(86) PCT No.: PCT/KR2023/016213

§ 371 Date: Apr. 27, 2026

(30) Foreign Application Priority Data
Oct. 20, 2022 (KR) 10-2022-0135724

(51) Int. Cl. A61F 5/56; A61B 5/00

(52) U.S. Cl. CPC A61F 5/566; A61B 5/4818; A61B 5/4836

(57) ABSTRACT

The present disclosure provides a sleep breathing disorder relief device capable of determining whether sleep breathing disorder occurs in a user and alleviating the sleep breathing disorder, wherein the sleep breathing disorder relief device includes: an oral unit mounted in an oral cavity of the user and moving a lower jaw of the user in response to whether the sleep breathing disorder occurs; a control unit receiving information measured while the user sleeps and controlling the oral unit to alleviate the sleep breathing disorder of the user according to the received information; and a sensing unit positioned between the control unit and the user's nasal cavity, and configured to sense airflow during the user's sleep to determine whether the sleep breathing disorder occurs.

### CROSS-REFERENCE TO RELATED APPLICATIONS

This application is a U.S. National Stage entry of International Patent Application no. PCT/KR2023/016213, filed Oct. 19, 2023, which claims the benefit of priority of Korean Patent Application no. KR10-2022-0135724, filed Oct. 20, 2022.

### BACKGROUND OF THE DISCLOSURE

### Technical Field

The present invention relates to a sleep breathing disorder relief device and a sleep breathing disorder relief system, and more particularly, the sleep breathing disorder relief device and the sleep breathing disorder relief system for determining whether the sleep breathing disorder by measuring air pressure and temperature according to a flow of air generated to respiration of a nasal cavity during sleep, and for relieving the sleep breathing disorder.

### Background Technology of the Invention

While sleeping, a person may take various actions without being aware of it, and it can be universally referred to as sleep talking or the sleep dialectic.

However, in addition to the general sleep talking or the sleep dialectic, there are things classified as sleep disorders even though they are various habitual behaviors that affect sleep, and in particular, there is a problem that sleep breathing disorder may directly endanger life as well as sleep quality.

Among them, snoring and sleep apnea symptoms may be caused by various causes, but may occur when the lower jaw is lowered by gravity and presses the airway, and when the intensity of snoring and sleep apnea symptoms increases, there may be problems in that the blood oxygen saturation decreases and suffocation may occur.

Therefore, snoring and sleep apnea symptoms require rapid treatment if they are severe, and it may lead to death, so a quick judgment may be important.

However, in the case of snoring and sleep apnea symptoms, it is difficult to accurately grasp the intensity or situation because they are symptoms that occur during sleep, and in order to properly grasp this, it is necessary to take a method of directly monitoring the sleeping state of the patient, and there may be disadvantages in that it is difficult to precisely control and treat because the patient cannot grasp his/her own state during sleep while receiving treatment.

In addition, among sleep breathing disorder, which is a symptom of snoring and sleep apnea, in the case of sleep apnea, no separate vibration or sound is generated, so unlike the case in which the symptoms of snoring can be reduced by determining the symptoms of snoring as vibration or sound, there may be a problem that it may be somewhat difficult to respond quickly because it is not possible to properly determine whether or not sleep apnea has occurred.

Therefore, various methods have been devised to determine whether sleep breathing disorder occurs and prevent the quality of sleep from deteriorating, and a means for solving the same is required.

### DESCRIPTION OF THE INVENTION

### Problems to be Solved of the Invention

The present invention has been made in an effort to solve the above-described problems of the prior art, and an object of the present invention is to measure an air pressure and a temperature according to a flow of air generated according to breathing of a nasal cavity in order to determine whether sleep breathing disorder occurs during sleep, and to alleviate sleep breathing disorder by determining whether sleep breathing disorder occurs through the measured air pressure and temperature.

The objects of the present invention are not limited to the above-mentioned objects, and other objects not mentioned will be clearly understood by those skilled in the art from the following description.

### Means for Solving Problems

In order to accomplish the above objects, the present disclosure provides a sleep breathing disorder relief device capable of determining whether sleep breathing disorder occurs in a user and alleviating the sleep breathing disorder, wherein the sleep breathing disorder relief device includes: an oral unit mounted in an oral cavity of the user and moving a lower jaw of the user in response to whether the sleep breathing disorder occurs; a control unit receiving information measured while the user sleeps and controlling the oral unit to alleviate the sleep breathing disorder of the user according to the received information; and a sensing unit positioned between the control unit and the user's nasal cavity, and configured to sense airflow during the user's sleep to determine whether the sleep breathing disorder occurs.

The sensing unit is disposed adjacent to the nasal cavity so as to sense at least one of the pressure and a temperature of air exhausted from the nasal cavity to determine whether the sleep breathing disorder occurs while the user sleeps.

The sensing unit includes a 1st sensing part for measuring air pressure due to the airflow of the nasal cavity and a 2nd sensing part for measuring the temperature of the airflow through the nasal cavity.

The 1st sensing part determines that the user has occurred the sleep breathing disorder when the measured air pressure is determined to be equal to or less than a first pressure, and transmits a first signal to the control unit.

The 1st sensing part determines that the user has not occurred p breathing disorder when the measured air pressure is determined to be equal to or more than a second pressure, and transmits a second signal to the control unit if the measured air pressure is determined to be between the first pressure and the second pressure.

The control unit determines that the sleep breathing disorder is in a predictive state when the second signal is transmitted from the first sensing part, and controls the oral unit.

The 2nd sensing part detects a temperature of the airflow through the nasal cavity and transmits a third signal to the control unit when the measured temperature of the airflow is equal to or lower than a preset temperature.

The control unit determines whether the user is sleep breathing disorder by comparing the third signal with information transferred from the 1st sensing part when the third signal is transferred through the 2nd sensing part.

The sensing unit further includes an 3rd sensing part configured to determine at least one of a distance or an angle between the 1st sensing part, the 2nd sensing part and the nasal cavity of the user, and transmit the determined distance or angle to the control unit, in order to prevent the measured air pressure and temperature from being lowered when the air pressure and temperature are measured in a state in which the angle between the 1st sensing part, the 2nd sensing part and the nasal cavity of the user is twisted.

The control unit corrects information transmitted from the 1st sensing part and the 2nd sensing part based on distance or angle transmitted from the 3re sensing part.

The sensing unit is located between the control unit and the nasal cavity of the user, is tiltably coupled to the control unit, and is tilted by the control unit according to a distance or an angle measured in the 3rd sensing part.

In addition, in order to accomplish the above objects, a sleep breathing disorder system for preventing sleep breathing disorder that occurs during sleep of a user, wherein the sleep breathing disorder relief system comprises: a sleep breathing disorder relief device; and a management server configured to share information transmitted from the sensing unit with the control unit, to determine whether the sleep breathing disorder occurs based on the information, to transmit the determination result to the control unit, and to continuously accumulate the information.

The management server is configured to calculate an average value of air pressure and temperature generated while the user sleeps based on the continuously accumulated information and transmit the calculated average value to the control unit.

The control unit receives an average value of air pressure and an average value of temperature transmitted from the management server and corrects the information transmitted from the sensing unit.

The control unit determines whether the sleep breathing disorder occurs in the user based on the information transferred from the 3rd sensing part and an average value of air pressure and temperature calculated from what has been accumulated in the management server.

### Effects of the Invention

In order to solve the above problems, the sleep breathing disorder relief device and the sleep breathing disorder relief system according to the present disclosure may measure an air pressure and a temperature according to a flow of air generated according to respiration of the nasal cavity in order to determine whether sleep breathing disorder occurs during sleep, and through this, it may be determined whether sleep breathing disorder occurs, thereby alleviating sleep breathing disorder.

The effects of the present invention are not limited to the effects mentioned above, and other effects not mentioned will be clearly understood by those skilled in the art from the description of the claims.

### DESCRIPTION OF DRAWINGS

The detailed description of the preferred embodiments of the present application described below, as well as the summary described above, will be better understood when read in connection with the accompanying drawings. Preferred embodiments are shown in the drawings for purposes of illustrating the present invention. However, it should be understood that the present application is not limited to the precise arrangements and means shown.

FIG. 1 is a diagram illustrating an overall description of a sleep breathing disorder relief device and a sleep breathing disorder relief system according to an embodiment of the present invention;

FIG. 2 is a diagram illustrating a configuration of an relief device of a sleep breathing disorder relief device and a sleep breathing disorder relief system according to an embodiment of the present invention;

FIG. 3 is a diagram illustrating a state in which an relief device of a sleep breathing disorder relief device and a sleep breathing disorder relief system is mounted according to an embodiment of the present invention;

FIG. 4 is a diagram illustrating an sensing unit of a sleep breathing disorder relief device and a sleep breathing disorder relief system according to an embodiment of the present invention;

FIG. 5 is a diagram illustrating a sleep breathing disorder relief device for charging a sleep breathing disorder relief system of an relief device and an charging device according to an embodiment of the present invention;

FIG. 6 is a diagram illustrating a sleep breathing disorder relief device of a sleep breathing disorder relief system and an ring unit according to an embodiment of the present invention;

FIG. 7 is a diagram illustrating an overall description of a sleep breathing disorder relief device system of a sleep breathing disorder relief system and an ring unit according to an embodiment of the present invention;

FIG. 8 is an illustrative drawing of an overall sleep breathing disorder system of a sleep breathing disorder relief device and sleep breathing disorder relief system according to one embodiment of the present invention;

FIG. 9 is a diagram illustrating a sleep breathing disorder relief device determination according to a signal of a sleep breathing disorder relief system and a sleep breathing disorder of a sleep breathing disorder relief device according to an embodiment of the present invention;

FIG. 10 is a diagram illustrating an assessment according to a signal of a sleep breathing disorder relief system and an 1st sensing part of an embodiment of the present invention; and

FIG. 11 is a diagram illustrating an control unit for measuring an myogram of a sleep breathing disorder relief device and a sleep breathing disorder relief system 2nd sensing part sleep breathing disorder relief device sleep breathing disorder relief system pad unit according to a variation of the present invention.

### SPECIFIC EMBODIMENTS FOR PRACTICING THE INVENTION

Hereinafter, a preferred embodiment of the present invention, in which an object of the present invention may be specifically realized, will be described with reference to the accompanying drawings.

In describing the present embodiment, the same names and the same reference numerals are used for the same components, and additional description thereof will be omitted.

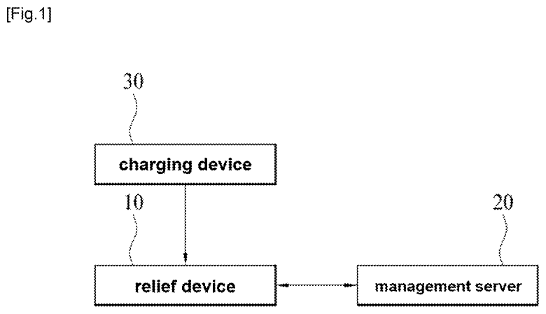

First, an overall description of the present invention may be given through FIG. 1. Specifically, FIG. 1 is a diagram for overall description of sleep breathing disorder relief device and sleep breathing disorder relief system according to an embodiment of the present invention.

Here, as shown in FIG. 1, according to an embodiment of the present invention, the mobile terminal may include a relief device 10 for alleviating the sleep breathing disorder depending on whether the user is in the relief device state, a management server 20 connected to the relief device 10 in real time to determine whether the sleep breathing disorder occurs in the relief device 10 together, and continuously accumulating and storing the determined information, and a charging device 30 for charging the FPCB 10.

In this case, the relief device 10 may include an oral unit 100 mounted in the oral cavity of the user to determine whether the sleep breathing disorder of the user occurs and move the lower jaw of the user according to whether the sleep breathing disorder occurs, an control unit 200 receiving information measured while the user sleeps and controlling the oral unit 100 to alleviate the sleep breathing disorder of the user according to the received information, and a sleep breathing disorder 300 provided between the control unit 200 and the nasal cavity of the user to sense information so as to determine whether the sleep breathing disorder sensing unit of the user occurs according to the flow of air inhaled while the user sleeps.

In addition, the relief device 10, which will be described later through the drawings, may further include a ring unit 400 mounted on at least one of the user's arms to determine whether the sleep breathing disorder is present or not according to a change in the blood oxygen saturation when the sleep breathing disorder is generated in the user.

Here, the oral unit 100 may be inserted into the oral cavity of the user, and in a state of being inserted into the oral cavity of the user, may control the mandible of the user and alleviate the sleep breathing disorder.

In addition, the sensing unit 300 may measure the flow of air according to whether the user breathes the nasal cavity except for the information of the ring unit 400 described above to more accurately determine whether the sleep breathing disorder is present, but may not be inserted into the nasal cavity of the user so as not to interfere with the sleep of the user, may measure the air pressure and temperature in a state of being provided adjacent to the nasal cavity, and may transmit the measured air pressure and temperature to the control unit 200.

Meanwhile, the control unit 200 may determine whether the sleep breathing disorder occurs to the user based on the information transmitted from the sensing unit 300 and the ring unit 400, and control the oral unit 100 according to the determined information to mitigate the sleep breathing disorder occurring to the user or may be connected to the management server 20 in real time to share the information transmitted from the sensing unit 300 and the ring unit 400.

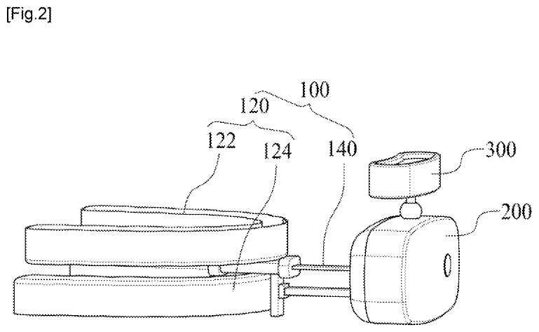

While the above-described configuration is more clearly described, FIGS. 2 to 7 may be referred to describe the utilization of the present invention.

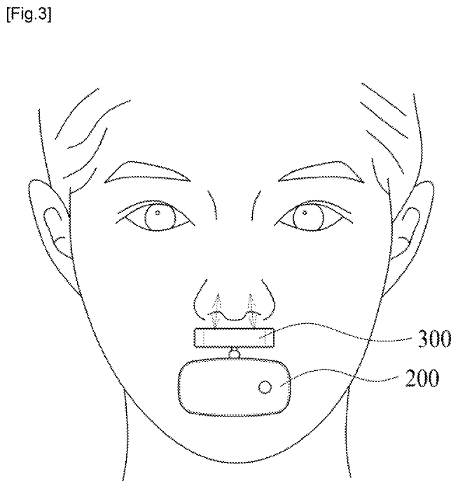

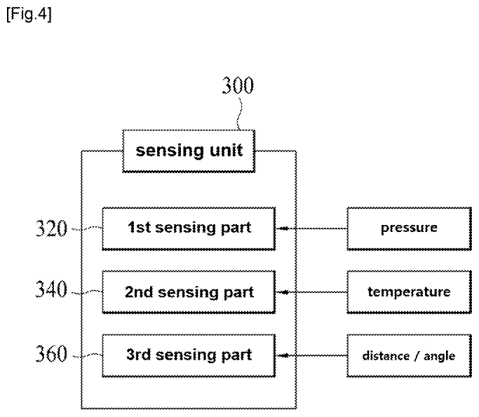

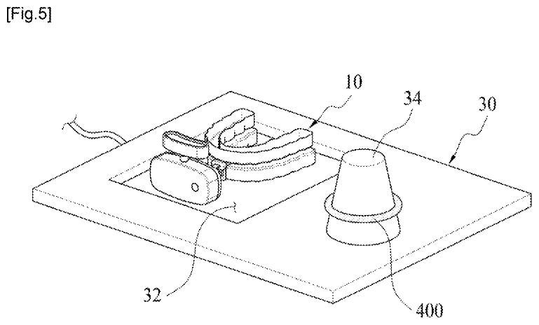

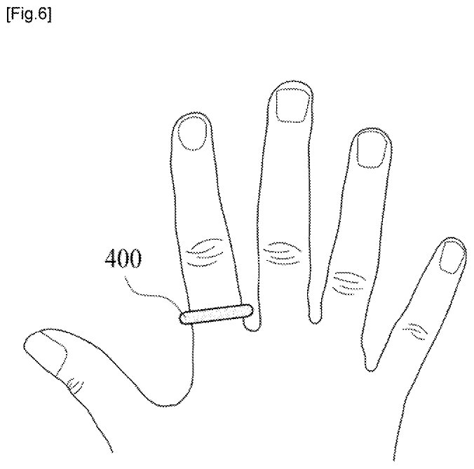

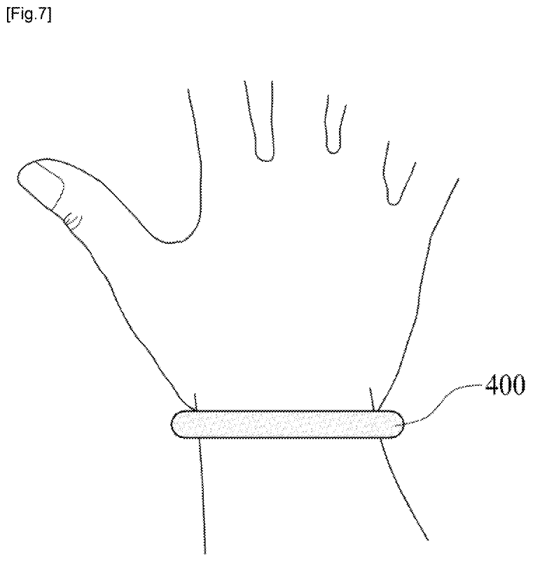

Specifically, FIG. 2 is a diagram for describing a configuration of an relief device of a sleep breathing disorder relief device and a sleep breathing disorder relief system according to an embodiment of the present disclosure, FIG. 3 is a diagram for describing a state in which an relief device of a sleep breathing disorder relief device and a sleep breathing disorder relief system is mounted according to an embodiment of the present disclosure, FIG. 4 is a diagram for describing an sensing unit of a sleep breathing disorder relief device and a sleep breathing disorder relief system according to an embodiment of the present disclosure, FIG. 5 is a diagram for describing a sleep breathing disorder relief system for charging an relief device of a sleep breathing disorder relief device and an charging device according to an embodiment of the present disclosure, FIG. 6 is a diagram for describing a sleep breathing disorder relief device of a sleep breathing disorder relief system and an ring unit according to an embodiment of the present disclosure, and FIG. 7 is a diagram for describing a sleep breathing disorder relief device of a sleep breathing disorder relief system and an ring unit according to a modified example of the present disclosure.

As shown in FIG. 2, the relief device 10 according to an embodiment of the disclosure may be divided into an oral unit 100, a control unit 200, a sensing unit 300, and a ring unit 400.

Here, the oral unit 100 may include a Mounting part 120 inserted into the oral cavity of the user and an adjustment part 140 connecting the Mounting part 120 and the control unit 200 and adjusting the position of the lower jaw of the user by adjusting the Mounting part 120.

In this case, the Mounting part 120 may be divided into a maxilla member 122 mounted on the user's upper jaw and a mandible member 124 mounted on the user's lower jaw to adjust a distance from the maxilla member 122.

In addition, the maxilla member 122 may be a reference of the user's upper jaw and may be fixed in position without additional adjustment of movement, and this may be generally caused by the sleep breathing disorder being pressed by the lower jaw, and thus the maxilla member 122 may fix the position.

In addition, as described above, the position of the mandible member 124 may be adjusted by the adjustment part 140, and as the position is adjusted based on the maxilla member 122, the mandible inserted into the mandible member 124 may press the airway of the user to release the sleep breathing disorder.

Meanwhile, the control unit 200 may determine whether to perform sleep breathing disorder based on information transferred from the sensing unit 300 to adjust the distance between the maxilla member 122 and the mandible member 124 through the adjustment part 140.

To this end, the control unit 200 may include a separate battery for supplying power while being mounted, a transceiver for transmitting and receiving data to and from the management server 20, and a determination unit for receiving information from the sensing unit 300 and determining the information to control the adjustment part 140, and it may be obvious that various essential components may be attached as necessary.

Meanwhile, the sensing unit 300 may include a 1st sensing part 320 for measuring an air pressure according to a flow of air generated according to a user's breathing state so as to determine whether sleep breathing disorder of the user is generated, a 2nd sensing part 340 for measuring a temperature of the air, a 1st sensing part 320 generated while an angle is twisted by determining a distance or an angle between the sensing unit 300 and a nasal cavity of the user, and a 3rd sensing part 360 for measuring a distance and an angle for correcting information of the 2nd sensing part 340.

This will be described in more detail with reference to the drawings to be described later.

As described above, the relief device 10 of the present disclosure may be used as illustrated in FIG. 3 such that the oral unit 100 is provided in a state of being inserted into the user's oral cavity and the sensing unit 300 is disposed at a position adjacent to the nasal cavity.

As described above, since the sensing unit 300 is not inserted into the nasal cavity of the user and is disposed only adjacent to the nasal cavity of the user to more clearly measure the information of the air pressure and the temperature, but is not inserted into the nasal cavity of the user, the quality of the sleep of the user may be prevented from being deteriorated by foreign body sensation.

In addition, basically, when the sensing unit 300 is disposed as illustrated in FIG. 3, the air pressure and the temperature may be more effectively measured in the case of exhalation than in the case of exhalation, and generally, when the sleep apnea symptom occurs in the sleep breathing disorder, since the air pressure and the temperature are not measured, it may be said that the air pressure and the temperature are not measured, and thus, it is possible to prevent the waste of power generated while measuring both exhalation and inhalation and to clearly measure the information measured through exhalation, thereby being more effective.

Meanwhile, as described above, the sensing unit 300 may include the 1st sensing part 320, the 2nd sensing part 340, and the 3rd sensing part 360 as illustrated in FIG. 4, and the 1st sensing part 320 may measure the air pressure, the 2nd sensing part 340 may measure the temperature, and the 3rd sensing part 360 may measure the distance or angle between the sensing unit 300 and the nasal cavity of the user.

Here, the information measured by the 1st sensing part 320, the 2nd sensing part 340, and the 3rd sensing part 360 is transferred to the control unit 200, and the control unit 200 may determine whether the user is sleep breathing disorder based on the information transferred from the 1st sensing part 320, the 2nd sensing part 340, and the 3rd sensing part 360.

In addition, as illustrated in FIG. 5, since the relief device 10 continuously senses information while the user sleeps, adjusts the lower jaw of the user, and determines whether the user is sleep breathing disorder, the RFID reader may be operated through a battery rather than being connected by a wire, and it may be necessary to charge power of the relief device 10 at a time point when the user is not sleeping.

However, since the battery charging through the electric wire requires a bonding portion to bond the electric wire to the relief device 10, and this is also the case in the ring unit 400, a plurality of electric wires may be used, which may cause more inconvenience, and thus, as illustrated in FIG. 5, the battery may be charged by wireless charging.

Here, the charging device 30 may be provided in a form of one panel, and may be divided into a 1st charging part 32 in which a groove is formed therein to allow the relief device 10 to be positioned and an 2nd charging part 34 in which the ring unit 400 is positioned, the 1st charging part 32 in which the relief device 10 is positioned may be formed to be depressed, and the 2nd charging part 34 in which the ring unit 400 is positioned may be formed to protrude so that the ring unit 400 is inserted, and although not necessarily limited thereto, providing a wireless charging space in the entire portion of the charging device 30 may be a waste of space and material, and thus it may be more effective to have such an arrangement relationship.

Meanwhile, the ring unit 400 may be formed in a ring shape inserted into any one of the fingers of the user as illustrated in FIG. 6, and through this, the blood oxygen saturation of the user may be measured and transmitted to the control unit 200.

Of course, a plurality of ring unit 400 may be provided to be inserted into a plurality of fingers rather than a single one, but this may cause a feeling of irritation or discomfort as in the sensing unit 300 described above, and thus it may be preferable to be provided in a single one.

Meanwhile, the ring unit 400 may be provided on a user's finger, but may also be provided in the form of a bracelet as shown in FIG. 7, which may be provided to mitigate the occurrence of an error due to the temperature or information of a finger that is separated from or adjacent to the user's finger, but it may be more preferable to provide it as shown in FIG. 6 in consideration of the simplicity of wearing.

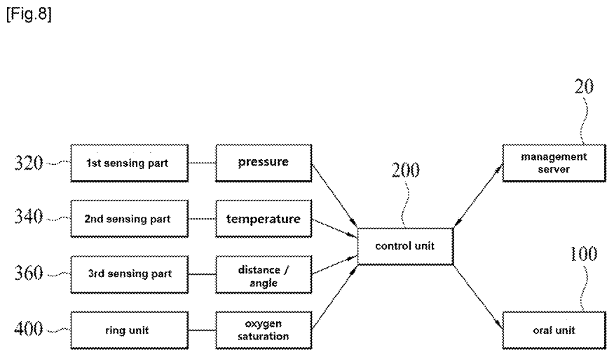

Based on the above-described configuration, FIGS. 8 to 10 may be referred to in order to describe the overall utilization of the present invention and the sleep breathing disorder system.

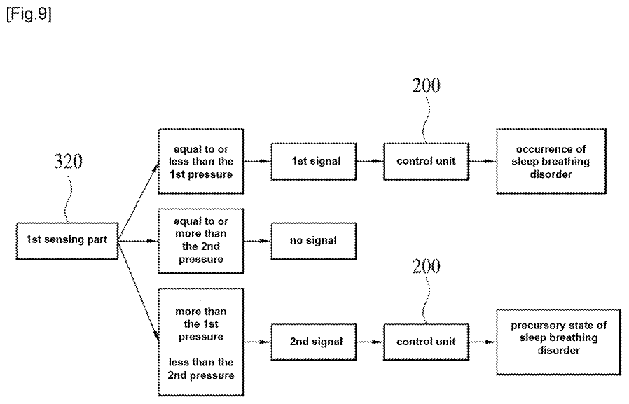

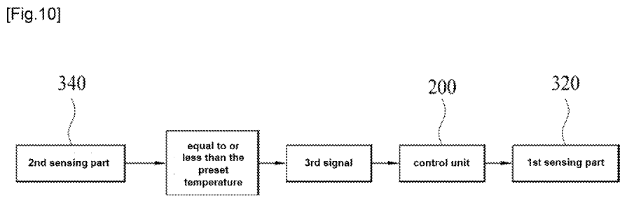

Specifically, FIG. 8 is a diagram for describing an overall sleep breathing disorder system of a sleep breathing disorder relief device and a sleep breathing disorder relief system according to an embodiment of the present invention, FIG. 9 is a diagram for describing control unit determination according to a signal of an 1st sensing part of a sleep breathing disorder relief device and a sleep breathing disorder relief system according to an embodiment of the present invention, and FIG. 10 is a diagram for describing determination according to a signal of an 2nd sensing part of a sleep breathing disorder relief device and a sleep breathing disorder relief system according to an embodiment of the present invention.

Referring to FIG. 8, the 1st sensing part 320 may measure an air pressure generated in an exhaled breath of a user and transmit the measured air pressure to the control unit 200, the 2nd sensing part 340 may determine a temperature of air generated in the exhaled breath of the user, the 3rd sensing part 360 may determine a distance or an angle between the sensing unit 300 and a nasal cavity of the user and transmit information about the determined distance or angle to the control unit 200, and the ring unit 400 may transmit information about a blood oxygen saturation of the user to the control unit 200.

The control unit 200 which has received the information on this may determine whether the sleep breathing disorder has occurred on the basis of the received information, and when it is determined that the sleep breathing disorder has occurred, may control the adjustment part 140 of the oral unit 100 to adjust the user's mandible to relieve the sleep breathing disorder, and the telegram transmitted to the control unit 200 may be transmitted to the management server 20 and stored and managed.

Here, the management server 20 may be connected to the external Internet in addition to simply storing information to check environmental information, and here, the environmental information may mean external temperature, internal temperature, humidity, precipitation, and the like.

The information of the air pressure transferred to the control unit 200 may be relatively corrected by the 3rd sensing part 360, but the temperature may vary depending on the internal and external temperatures and thus correction may be required, and thus the information transferred may be corrected when the control unit 200 determines by checking the information.

In addition, the control unit 200 may also correct information such as air pressure, temperature, oxygen saturation, and the like transmitted through the environment information transmitted from the management server 20, and in addition, the degree of torsion between the sensing unit 300 and the nasal cavity of the user may be determined through the angle and distance information transmitted from the 3rd sensing part 360, and the distance between the sensing unit 300 and the nasal cavity of the user may be directly tilted and adjusted, or the air pressure and temperature information may be corrected and determined using the angle and distance information.

That is, the control unit 200 may more clearly determine whether the sleep breathing disorder occurs based on the environment information transferred from the management server 20 and the angle and distance information transferred from the 3rd sensing part 360.

Meanwhile, as illustrated in FIG. 9, the 1st sensing part 320 that transmits information to the control unit 200 may transmit different signals to the control unit 200 according to the case of the first pressure to the second pressure when the measured air pressure is equal to or less than the first pressure and equal to or greater than the second pressure.

For example, when it is determined that the pressure is equal to or lower than the first pressure, that is, when the air pressure of the user is lowered to a predetermined level or lower and the possibility of the sleep breathing disorder state is considerably high, the first signal is transmitted to the control unit 200, and when it is determined that the first signal is appropriate through the above-described various information corrections, the control unit 200 may determine that the sleep breathing disorder has occurred and control the lower jaw of the user through the adjustment part 140.

However, when the air pressure is equal to or greater than the second pressure, that is, equal to or greater than a predetermined level, since the breathing is normally performed, it is determined that the possibility of occurrence of sleep breathing disorder is remarkably low, and thus a separate signal may not be transmitted to the control unit 200.

Meanwhile, when the pressure is equal to or higher than the first pressure but lower than the second pressure, the second signal is transmitted to the control unit 200, and when it is determined that there is no abnormality in the second signal even after correction, the control unit 200 may determine that the user is in a precursor state before the sleep breathing disorder occurs, and may move the user's lower jaw by a predetermined interval.

This is more effective to cope with and prevent the problem before simply dealing with the problem rather than immediately after the problem occurs, and when the airway is blocked by the mandible causing sleep apnea, the movement distance of the mandible increases, which disturbs the sleep of the user such as waking up or turning over, so to prevent this, it is possible to determine the precursor state of sleep breathing disorder and adjust the mandible at a predetermined interval.

Meanwhile, when it is determined that the measured temperature is equal to or lower than a preset temperature, that is, while breathing continues, since it is not clear whether the temperature is sleep breathing disorder or not due to remaining heat, the 2nd sensing part 340 may more clearly transmit the third signal to the control unit 200 at a temperature equal to or lower than the preset temperature.

Accordingly, rather than simply controlling the adjustment part 140 depending on only the information of the 2nd sensing part 340, the control unit 200 may determine whether the information is transferred from the 1st sensing part 320, and when the information is transferred from the 1st sensing part 320, may determine whether the sleep breathing disorder has occurred by comparing the information of the 1st sensing part 320 with the information of the 2nd sensing part 340.

That is, according to an embodiment of the present disclosure, the control unit 200 may alleviate and prevent the sleep breathing disorder by adjusting the user's lower jaw based on the information transmitted from the sensing unit 300, and may make a more clear determination based on the information of the 3rd sensing part 360 that determines the torsion between the sensing unit 300 and the user's nasal cavity and the environment information transmitted from the management server 20 in order to determine the accuracy of the transmitted information, and in addition, may make it possible to make it more clear whether the sleep breathing disorder occurs during sleep by inputting a life pattern through a separate device.

In addition, the control unit 200 may correct information on the first pressure, the second pressure, and the preset temperature on the basis of the information on the 3rd sensing part 360 and the information on the management server 20 so that the first signal, the second signal, and the third signal may be more clearly transmitted, thereby more clearly determining whether to perform sleep breathing disorder and preventing sleep breathing disorder in advance in the headlight state.

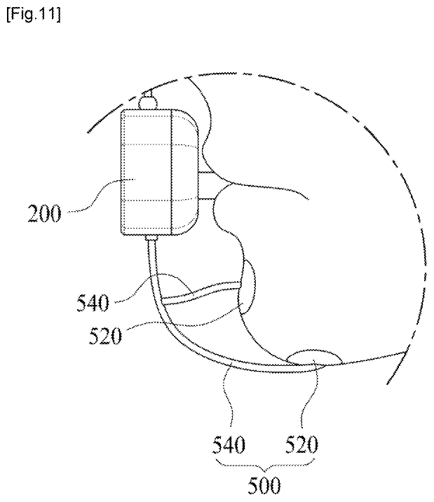

Meanwhile, in the present invention, the pad unit 500 may be provided in order to more accurately determine sleep breathing disorder such as snoring or bruxism while determining whether the user is sleeping apnea, which may be described with reference to FIG. 11.

Specifically, FIG. 11 is a diagram illustrating pad unit for measuring electromyogram of sleep breathing disorder relief device and sleep breathing disorder relief system according to a modified example of the present invention.

First, as shown in FIG. 11, in order to measure electromyogram, it is possible to measure the electromyogram through the user's chin part, and to this end, it may include an attachment part 520 for measuring the electromyogram at the chin part and a connecting part 540 for connecting the attachment part 520 and the control unit 200.

Here, the electromyogram measured through the attachment part 520 may measure tremors or movements of the jaw, and information on the tremors or movements may be transmitted to the control unit 200, so that the type of sleep breathing disorder generated by the user in the control unit 200 may be more widely identified.

In addition, although FIG. 11 illustrates that a pair is attached to the bottom of the chin and the bottom of the lips, the pair may be attached only to the bottom of the chin or may be individually attached only to the bottom of the lips, but is not necessarily limited thereto, and it may be preferable that a pair is provided as illustrated in FIG. 11 for accuracy of transmitted information.

Preferred embodiments according to the present invention have been described, and it is obvious to those skilled in the art that the present invention may be embodied in other specific forms without departing from the spirit or scope thereof in addition to the above-described embodiments.

Therefore, the embodiments described above should be regarded as illustrative rather than restrictive, and accordingly, the present invention is not limited to the above description and may be changed within the scope of the appended claims and their equivalents.

### EXPLANATION OF SYMBOLS

10: relief device
20: management server
30: charging device
32: 1st charging part
34: 2nd charging part
100: oral unit
120: Mounting part
122: maxilla member
124: mandible member
140: adjustment part
200: control unit
300: sensing unit
320: 1st sensing part
340: 2nd sensing part
360: 3rd sensing part
400: ring unit
500: pad unit
520: attachment part
540: connecting part

1. A sleep breathing disorder relief device capable of determining whether sleep breathing disorder occurs in a user and alleviating the sleep breathing disorder,
wherein the sleep breathing disorder relief device includes:
an oral unit mounted in an oral cavity of the user and moving a lower jaw of the user in response to whether the sleep breathing disorder occurs;
a control unit receiving information measured while the user sleeps and controlling the oral unit to alleviate the sleep breathing disorder of the user according to the received information; and
a sensing unit positioned between the control unit and the user's nasal cavity, and configured to sense airflow during the user's sleep to determine whether the sleep breathing disorder occurs.

2. The sleep breathing disorder relief device of claim 1, wherein the sensing unit is disposed adjacent to the nasal cavity so as to sense at least one of the pressure and a temperature of air exhausted from the nasal cavity to determine whether the sleep breathing disorder occurs while the user sleeps.

3. The sleep breathing disorder relief device of claim 2, wherein the sensing unit includes a 1st sensing part for measuring air pressure due to the airflow of the nasal cavity and a 2nd sensing part for measuring the temperature of the airflow through the nasal cavity.

4. The sleep breathing disorder relief device of claim 3, wherein the 1st sensing part determines that the user has occurred the sleep breathing disorder when the measured air pressure is determined to be equal to or less than a first pressure, and transmits a first signal to the control unit.

5. The sleep breathing disorder relief device of claim 4, wherein the 1st sensing part determines that the user has not occurred p breathing disorder when the measured air pressure is determined to be equal to or more than a second pressure, and transmits a second signal to the control unit if the measured air pressure is determined to be between the first pressure and the second pressure.

6. The sleep breathing disorder relief device of claim 5, wherein the control unit determines that the sleep breathing disorder is in a predictive state when the second signal is transmitted from the first sensing part, and controls the oral unit.

7. The sleep breathing disorder relief device of claim 3, wherein the 2nd sensing part detects a temperature of the airflow through the nasal cavity and transmits a third signal to the control unit when the measured temperature of the airflow is equal to or lower than a preset temperature.

8. The sleep breathing disorder relief device of claim 7, wherein the control unit determines whether the user is sleep breathing disorder by comparing the third signal with information transferred from the 1st sensing part when the third signal is transferred through the 2nd sensing part.

9. The sleep breathing disorder relief device of claim 3, wherein the sensing unit further includes an 3rd sensing part configured to determine at least one of a distance or an angle between the 1st sensing part, the 2nd sensing part and the nasal cavity of the user, and transmit the determined distance or angle to the control unit, in order to prevent the measured air pressure and temperature from being lowered when the air pressure and temperature are measured in a state in which the angle between the 1st sensing part, the 2nd sensing part and the nasal cavity of the user is twisted.

10. The sleep breathing disorder relief device of claim 9, wherein the control unit corrects information transmitted from the 1st sensing part and the 2nd sensing part based on distance or angle transmitted from the 3rd sensing part.

11. The sleep breathing disorder relief device of claim 9, wherein the sensing unit is located between the control unit and the nasal cavity of the user, is tiltably coupled to the control unit, and is tilted by the control unit according to a distance or an angle measured in the 3rd sensing part.

12. A sleep breathing disorder system for preventing sleep breathing disorder that occurs during sleep of a user,
wherein the sleep breathing disorder relief system comprises:
a sleep breathing disorder relief device of claim 1; and
a management server configured to share information transmitted from the sensing unit with the control unit, to determine whether the sleep breathing disorder occurs based on the information, to transmit the determination result to the control unit, and to continuously accumulate the information.

13. The sleep breathing disorder relief system of claim 12, wherein the management server is configured to calculate an average value of air pressure and temperature generated while the user sleeps based on the continuously accumulated information and transmit the calculated average value to the control unit.

14. The sleep breathing disorder relief system according to claim 13, wherein the control unit receives an average value of air pressure and an average value of temperature transmitted from the management server and corrects the information transmitted from the sensing unit.

15. The sleep breathing disorder relief system of claim 13, wherein the control unit determines whether the sleep breathing disorder occurs in the user based on the information transferred from the 3rd sensing part and an average value of air pressure and temperature calculated from what has been accumulated in the management server.
